(19) United States
(12) Patent Application Publication
(10) Pub. No.: US 2012/0209069 A1
(43) Pub. Date: Aug. 16, 2012

(54) COLLISION AVOIDANCE AND DETECTION USING DISTANCE SENSORS

(75) Inventors: Aleksandra Popovic, New york, NY (US); Mareike Klee, Straelen (DE); Bout Marcelis, Eindhoven (NL); Christianus Martinus Van Heesch, Eindhoven (NL)

(73) Assignee: KONINKLIJKE PHILIPS ELECTRONICS N.V., EINDHOVEN (NL)

(21) Appl. No.: 13/502,412

(22) PCT Filed: Oct. 4, 2010
(86) PCT No.: PCT/IB10/54481

§ 371 Date: Apr. 17, 2012

Related U.S. Application Data
(60) Provisional application No. 61/257,857, filed on Nov. 4, 2009.

(51) Int. Cl. A61B 1/00; A61B 1/04

(52) U.S. Cl. 600/109; 600/118

(57) ABSTRACT

An endoscopic method involves an advancement of an endoscope (20) as controlled by an endoscopic robot (31) to a target location within an anatomical region of a body, and a generation of a plurality of monocular endoscopic images (80) of the anatomical region as the endoscope (20) is advanced to the target location by the endoscopic robot (31). For avoiding or detecting a collision of the endoscope (20) with and object within monocular endoscopic images (80) (e.g., a ligament within monocular endoscopic images of a knee), the method further involves a generation of distance measurements of the endoscope (20) from the object as the endoscope (20) is advanced to the target location by the endoscopic robot (31), and a reconstruction of a three-dimensional image of a surface of the object within the monocular endoscopic images (80) as a function of the distance measurements (81).

The present invention generally relates to minimally invasive surgeries involving an endoscope manipulated by an endoscopic robot. The present invention specifically relates to avoiding and detecting a collision by an endoscope using distance sensors with an object within an anatomical region of a body and a reconstruction of the surface imaged by the endoscope.

Generally, a minimally invasive surgery utilizes an endoscope, which is a long, flexible or rigid tube having an imaging capability. Upon insertion into a body through a natural orifice or a small incision, the endoscope provides an image of the region of interest that may be viewed through an eyepiece or on a screen as a surgeon performs the operation. Essential to the surgery is the depth information of object(s) within the image that will enable the surgeon to be able to advance the endoscope while avoiding the object(s). However, the frames of an endoscopic image are two-dimensional and the surgeon therefore may lose the perception of the depth of object(s) viewed in the screen shot of the image.

More particularly, rigid endoscopes are used to provide visual feedback during major types of minimally invasive procedures including, but not limited to, endoscopic procedures for cardiac surgery, laparoscopic procedures for the abdomen, endoscopic procedures for the spine and arthroscopic procedures for joints (e.g., a knee). During such procedures, a surgeon may use an active endoscopic robot for moving the endoscope autonomously or by commands from the surgeon. In either case, the endoscopic robot should be able to avoid collision of the endoscope with important objects within the region of interest in the patient's body. Such collision avoidance may be difficult for procedures involving real-time changes in the operating site (e.g., real-time changes in a knee during ACL arthroscopy due to removal of damaged ligament, repair of menisci and/or a drilling of a channel), and/or different positioning of the patient's body during surgery than in preoperative imaging (e.g., knee is straight during a preoperative computer-tomography and is bent during the surgery).

The present invention provides a technique that utilizes endoscopic video frames from the monocular endoscopic images and distance measurements of an object within the monocular endoscopic images to reconstruct a 3D image of a surface of an object viewed by the endoscope for the purposes of avoiding and detecting any collision by an endoscope with the object.

One form of the present invention is a endoscopic system employing an endoscope and an endoscopic control unit having an endoscopic robot. In operation, the endoscope generates a plurality of monocular endoscopic images of an anatomical region of a body as the endoscope is advanced by the endoscopic robot to a target location within the anatomical region. Additionally, the endoscope includes one or more distance sensors for generating measurements of a distance of the endoscope from an object within the monocular endoscopic images as the endoscope is advanced to the target location by the endoscopic robot (e.g., distance to a ligament within monocular endoscopic images of a knee). For avoiding or detecting a collision of the endoscope with the object, the endoscopic control unit receives the monocular endoscopic images and distance measurements to reconstruct a three-dimensional image of a surface of the object within the monocular endoscopic images as a function of the distance measurements.

A second form of the present invention is an endoscopic method involving an advancement of an endoscope by an endoscopic robot to a target location within an anatomical region of a body and a generation of a plurality of monocular endoscopic images of the anatomical region as the endoscope is advanced by the endoscopic robot to the target location within the anatomical region. For avoiding or detecting a collision of the endoscope with an object within the monocular endoscopic images (e.g., a ligament within monocular endoscopic images of a knee), the method further involves a generation of distance measurements of the endoscope from the object as the endoscope is advanced to the target location by the endoscopic robot, and a reconstruction of a three-dimensional image of a surface of the object within the monocular endoscopic images as a function of the distance measurements.

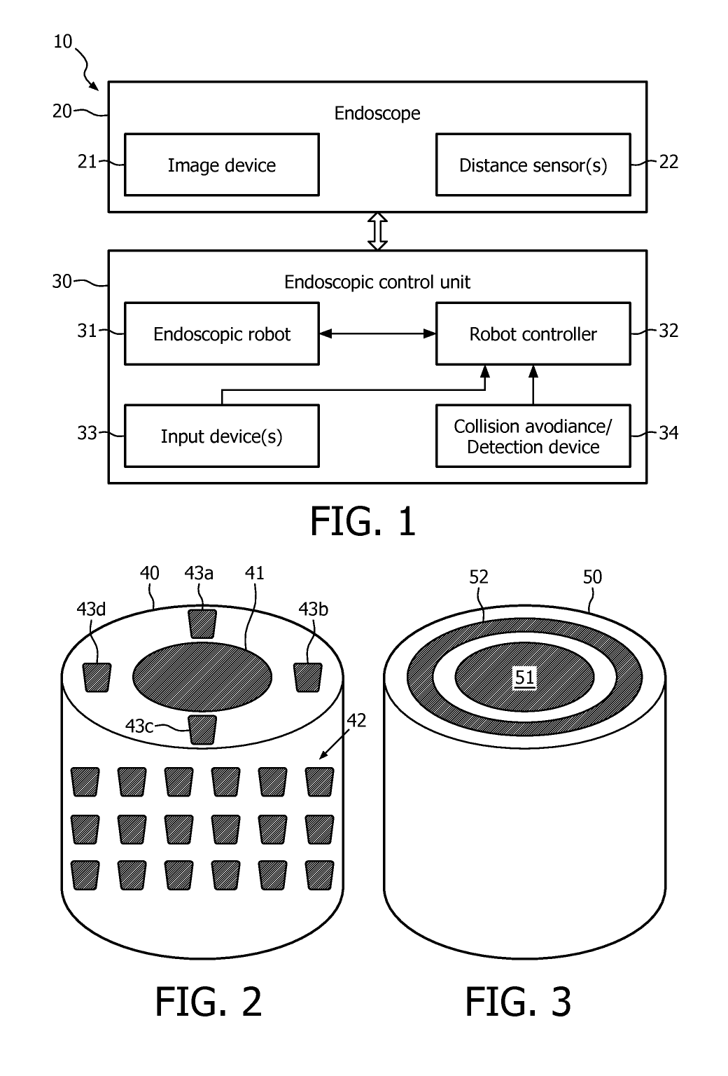

FIG. 1. illustrates an exemplary embodiment of a endoscopic system in accordance with the present invention.

FIG. 2 illustrates a first exemplary embodiment of a distal end of an endoscope in accordance with the present invention.

FIG. 3 illustrates a second exemplary embodiment of a distal end of an endoscope in accordance with the present invention.

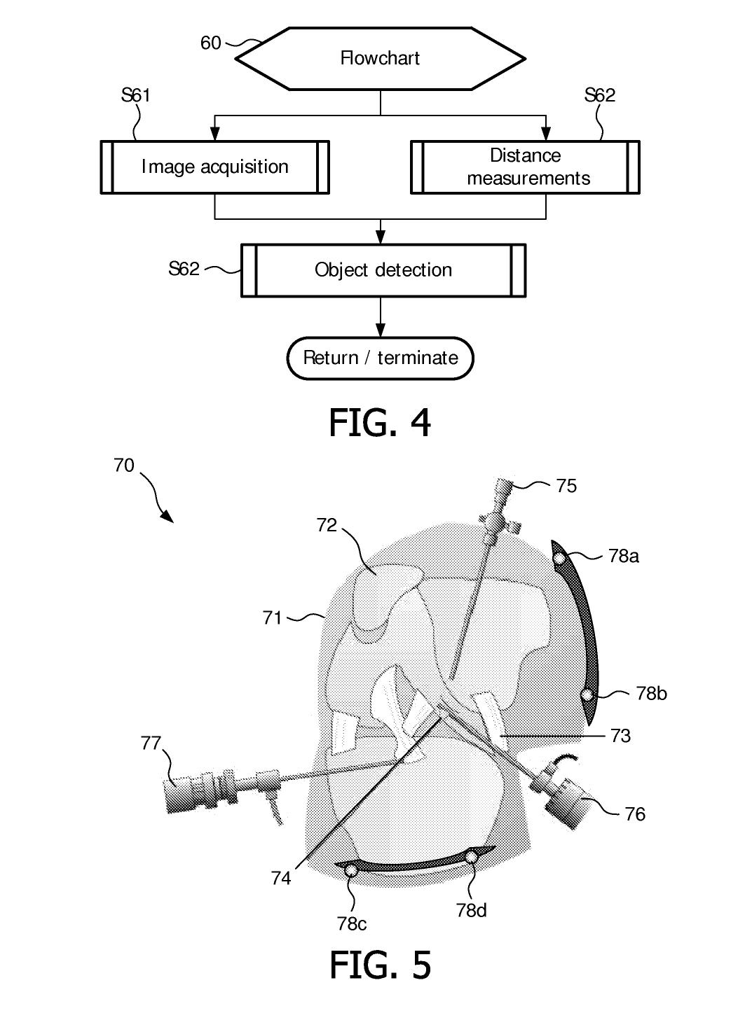

FIG. 4 illustrates a flowchart representative of an exemplary embodiment of a collision avoidance/detection method in accordance with the present invention.

FIG. 5 illustrates a schematic representation of an arthroscopic surgery in accordance with the present invention.

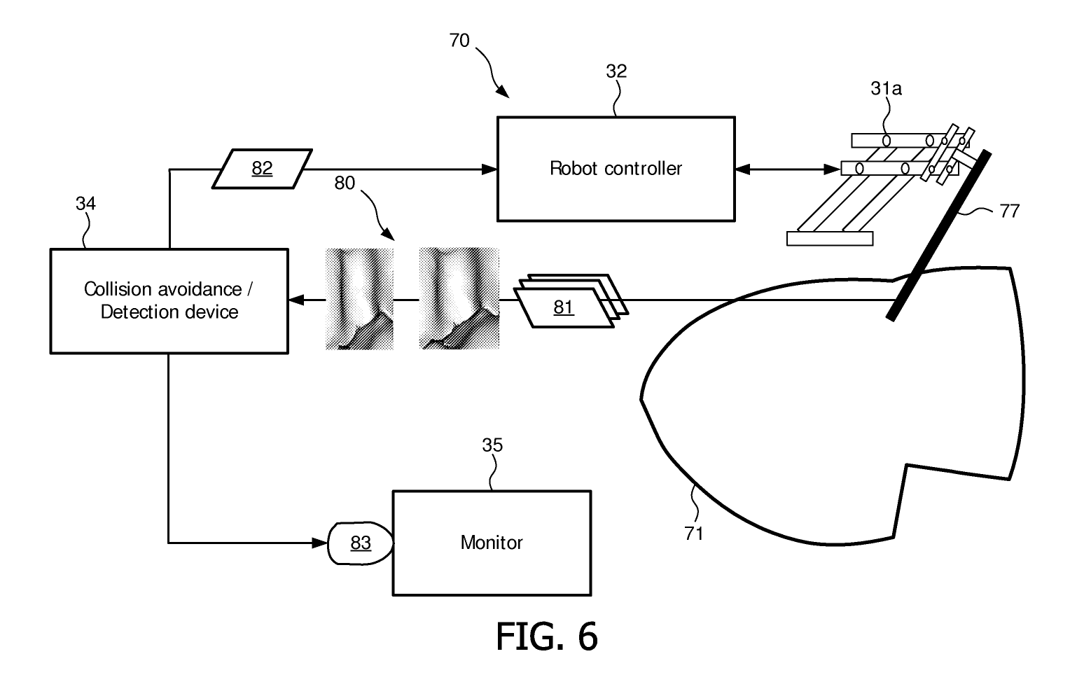

FIG. 6 illustrates an exemplary application of the flowchart illustrated in FIG. 4 during the arthroscopic surgery illustrated in FIG. 5.

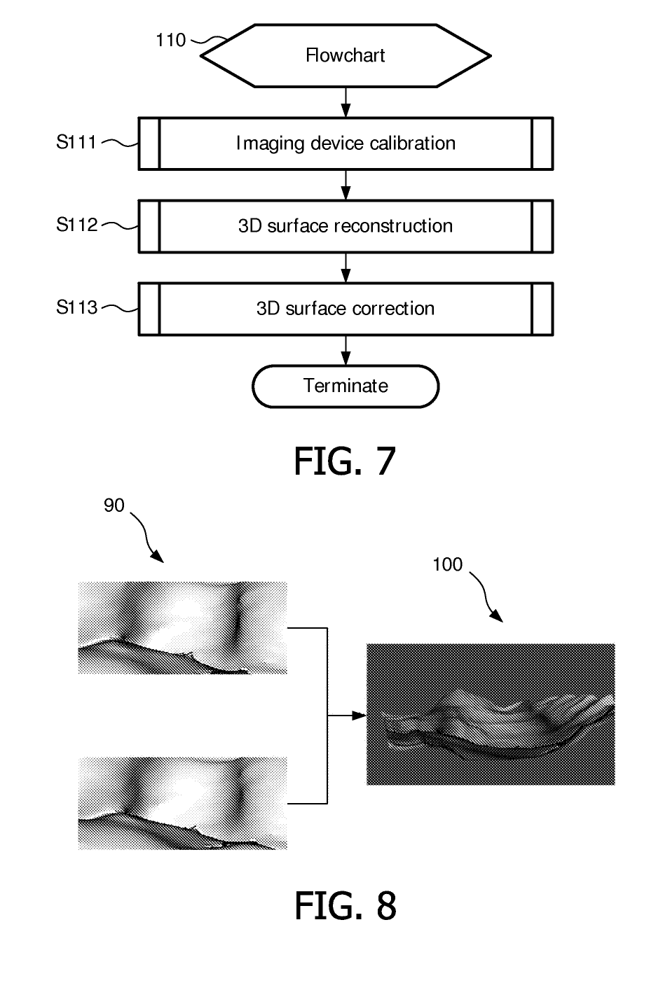

FIG. 7 illustrates a flowchart representative of an exemplary embodiment of an object detection in accordance with the present invention.

FIG. 8 illustrates an exemplary stereo matching of two synthetic knee images in accordance with the present invention.

As shown in FIG. 1, a endoscopic system 10 of the present invention employs an endoscope 20 and a endoscopic control unit 30 for any applicable type of medical procedures. Examples of such medical procedures include, but are not limited to, minimally invasive cardiac surgery (e.g., coronary artery bypass grafting or mitral valve replacement), minimally invasive abdominal surgery (laparoscopy) (e.g., prostatectomy or cholecystectomy, and natural orifice translumenal endoscopic surgery.

Endoscope 20 is broadly defined herein as any device structurally configured imaging an anatomical region of a body (e.g., human or animal) via an imaging device 21 (e.g., fiber optics, lenses, miniaturized CCD based imaging systems, etc). Examples of endoscope 20 include, but are not limited to, any type of imaging scope (e.g., a bronchoscope, a colonoscope, a laparoscope, an arthroscope, etc.) and any device similar to a scope that is equipped with an image system (e.g., an imaging cannula).

Endoscope 20 is further equipped on its distal end with one or more distance sensors 22 as individual element(s) or array(s). In one exemplary embodiment, a distance sensor 22 may be an ultrasound transducer element or array for transmitting and receiving ultrasound signals having a time of flight that is indicative of a distance to an object (e.g., a bone within a knee). The ultrasound transducer element/array may be thin film micro-machined (e.g., piezoelectric thin film or capacitive micro-machined) transducers, which may also be disposable. In particular, a capacitive micro-machined ultrasound transducer array has AC characteristics for time of flight distance measurement of an object, and DC characteristics for direct measurement of any pressure being exerted by the object of the membrane of the array.

In practice, distance sensor(s) 22 are located on a distal end of endoscope 20 relative to imaging device 21 to facilitate collision avoidance and detection by endoscope 20 with an object. In one exemplary embodiment as shown in FIG. 2, distance sensors in the form of ultrasound transducer array 42 and ultrasound transducer array 43 are positioned around a circumference and a front surface, respectively, of a distal end of an endoscope shaft 40 having a imaging device 41 on the front surface of its distal end. For this embodiment, arrays 42 and 43 provide sensing around a significant length of endoscope shaft 40. By making use 1D or 2D ultrasound transducer arrays, steering of the ultrasound beam in an angle of +/−45 degree to transmit and receive ultrasound signals is obtain whereby objects positioned in the direct line of the ultrasound sensors as well as objects located under an angle may be detected and collision with these objects may be avoided.

In another exemplary embodiment as shown in FIG. 3, a distance sensor in the form of a single ultrasound linear element 52 encircles a imaging device 51 on a top distal end of an endoscope shaft 50. Alternatively, ultrasound linear element 52 may consist of several elements serving as a phase-array for beam-forming and beam-steering.

Referring again to FIG. 1, endoscopic robot 31 of unit 30 is broadly defined herein as any robotic device structurally configured with motorized control to maneuver endoscope 20 during a minimally invasive surgery, and robot controller 32 of unit 30 is broadly defined herein as any controller structurally configured to provide motor signals to endoscopic robot 31 for the purposes of maneuvering endoscope 20 during the minimally invasive surgery. Exemplary input device(s) 33 for robot controller 32 include, but are not limited to, a 2D/3D mouse and a joystick.

Collision avoidance/detection device 34 of unit 30 is broadly defined herein as any device structurally configured for providing a surgeon operating an endoscope or a endoscopic robot with a real-time collision avoidance/detection by endoscope 20 with an object within an anatomical region of a body using a combination of imaging device 21 and distance sensors 22. In practice, collision avoidance/detection device 34 may operate independently of robot controller 32 as shown or be internally incorporated within robot controller 32.

Flowchart 60 as shown in FIG. 4 represents a collision avoidance/detection method of the present invention as executed by collision avoidance/detection device 34. For this method, collision avoidance/detection device 34 initially executes a stage S61 for acquiring monocular endoscopic images of an object within the anatomical region of a body from imaging device 21, and a stage S62 for receiving distance measurements of endoscope 20 from the object from distance sensor(s) 22 while endoscope 20 is advanced to a target location within the anatomical region of the body by endoscopic robot 31. From the image acquisition and distance measurements, collision avoidance/detection device 34 proceeds to a stage S63 of flowchart 60 to detect the object whereby the surgeon may manually operate endoscopic robot 31 or endoscopic robot 31 may be autonomously operated to avoid or detect any collision by endoscope 20 with the object. The detection of the object involves a 3D reconstruction of a surface of the object as viewed by endoscope 20 that provides critical information for avoiding and detecting any collision by endoscope with the object including, but not limited to, a 3D shape of the object and a depth of every point on the surface of the object.

To facilitate an understanding of flowchart 60, stages S61-S63 will now be described in more detail in the context of an arthroscopic surgical procedure 70 as shown in FIGS. 5 and 6. Specifically, FIG. 5 illustrates a patella 72, a ligament 73 and a damaged cartilage 74 of a knee 71. A irrigating instrument 75, a trimming instrument 76 and an arthroscope 77 having an imaging device in the form of a imaging device (not shown) and a distance sensor in the form of an ultrasound transducer array (not shown) are being used for purposes of repairing the damaged cartilage 74. Also, illustrated are ultrasound transducers 78a-78d for determining a relative positioning of the ultrasound transducer array within knee 71.

FIG. 6 illustrates a control of arthroscope 77 by an endoscopic robot 31a.

Referring to FIG. 4, the image acquisition of stage S61 involves the imaging device of arthroscope 77 providing a two-dimensional image temporal sequence 80 (FIG. 6) to collision avoidance/detection device 34 as arthroscope 77 is being advanced to a target location within knee 71 by endoscopic robot 31a as controlled by robot controller 32. Alternatively, the ultrasound transducer array of arthroscope 77 may be utilized to provide two-dimensional temporal sequence 90.

The distance measurements of stage S62 involve the ultrasound transducer array of arthroscope 77 transmitting and receiving ultrasound signals within knee 71 having a time of flight that is indicative of a distance to an object and provides collision avoidance/detection device 34 with distance measurement signals 81 (FIG. 6). In one embodiment, distance measurement signals may have AC signal components for time of flight distance measurement of an object, and DC signal components for direct measurement of any pressure being exerted by the object of the membrane of the ultrasound transducer array.

The object depth estimation of stage S63 involves collision avoidance/detection device 34 using a combination of image temporal sequence 80 and distance measurement signals 81 to provide control signals 82 to robot controller 32 and/or display image data 83 to a monitor 35 as needed to enable a surgeon or endoscopic robot 31 to avoid the object or to maneuver away from the object in the case of a collision. The display of image data 93 further provides information for facilitating the surgeon in making any necessary intraoperative decisions, particularly the 3D shape of the object and the depth of each point on the surface of the object.

Flowchart 110 as shown in FIG. 7 represents an exemplary embodiment of stage S63 (FIG. 4). Specifically, the detection of the object by device 34 is achieved by an implementation of a multiple stereo matching algorithm based on epipolar geometry.

First, a calibration of imaging device is executed during a stage S111 of flowchart 110 prior to an insertion of arthroscope 77 within knee 71. In one embodiment of stage S111, a standardized checkerboard method may be used to obtain intrinsic imaging device parameters (e.g., focal point and lens distortion coefficients) in a 3×3 imaging device intrinsic matrix (K).

Second, as arthroscope 77 is being advanced to a target location within knee 71, a reconstruction of a 3D surface of an object from two or more images of the same scene taken at different time moments is executed during a stage S112 of flowchart 110. Specifically, motion of endoscope 71 is known from control of endoscopic robot 31, so a relative rotation (3×3 matrix R) and a translation (3×1 vector t) between the two respective imaging device positions is also known. Using a knowledge set (K,R,t), comprising of both intrinsic and extrinsic imaging device parameters, image rectification is implemented to build a 3D depth map from the two images. In this process, the (K,R,t) images are warped so that their vertical components are aligned. The process of rectification results in 3×3 warping matrices and 4×3 disparity-to-depth mapping matrix.

Next, an optical flow is computed between two images during stage S112, using point correspondences as known in the art. Specifically, optical flow (u,v) in each 2D point (x,y) represents points movement between two images. Since the images are rectified, (i.e. warped to be parallel), then v=0. Finally, from optical flow, a disparity map in every image element is u (x1−x2). Re-projecting the disparity map using the 4×3 disparity-to-depth mapping matrix will result in the 3D shape of the object in front of the lens of the imaging device. FIG. 8 illustrates an exemplary result of a 3D surface reconstruction 100 from image temporal sequence 80.

It is possible to detect distance between the lens and other structures. However, given an immeasurable imperfections in image temporal sequence 80 and any discretization errors, a stage S113 of flowchart 110 is implemented to correct the 3D surface reconstruction as needed. The correction starts with a comparison of the depth(s), dₛᵢ, i=1, . . . , N measured by N (one or more) distance sensors 22 and depth(s) dᵢᵢ i=1, . . . , N measured from the reconstructed images. These distances should be the same, however, because of the measurement noises, each of N measurement position will have an error associated with it: eᵢ=|dₛᵢ−dᵢᵢ|, i=1, . . . , N. The direct measurement using distance sensors 22 is significantly more precise than image- based method. Image-based method has however denser measurement. Therefore, the set eᵢ is used to perform an elastic warping of the reconstructed surface to improve precision.

Although the present invention has been described with reference to exemplary aspects, features and implementations, the disclosed systems and methods are not limited to such exemplary aspects, features and/or implementations. Rather, as will be readily apparent to persons skilled in the art from the description provided herein, the disclosed systems and methods are susceptible to modifications, alterations and enhancements without departing from the spirit or scope of the present invention. Accordingly, the present invention expressly encompasses such modification, alterations and enhancements within the scope hereof.

1. An endoscopic system (10), comprising:
an endoscope (20) for generating a plurality of monocular endoscopic images (80) of an anatomical region (71) of a body as the endoscope (20) is advanced to a target location within the anatomical region (71),
wherein the endoscope (20) includes at least one distance sensor (22) for generating measurements (81) of a distance of the endoscope (20) from an object within the monocular endoscopic images (80) as the endoscope (20) is advanced to the target location; and
an endoscopic control unit (30) in communication with the endoscope (20) to receive the monocular endoscopic images (80) and the distance measurements (81),
wherein the endoscopic control unit (30) includes an endoscopic robot (31) operable to advance the endoscope (20) to the target location, and
wherein the endoscopic control unit (30) is operable to reconstruct a three-dimensional image of a surface of the object within the monocular endoscopic images (80) as a function of the distance measurements (81).

2. The endoscopic system (10) of claim 1, wherein the reconstruction of the three-dimensional image of the surface of the object includes:
building a three-dimensional depth map of the object from a temporal sequence of the monocular endoscopic images (80) of the anatomical region (71); and
correcting the three-dimensional depth map of the object relative to at least two distance measurements, each distance measurement being associated with one of the monocular endoscopic images.

3. The endoscopic system (10) of claim 2, wherein the correction of the three-dimensional image of the surface of the object includes:
generating an error set representative of a comparison of the depth map to a depth of each point of a surface of the object as indicated by the at least two distance measurements.

4. The endoscopic system (10) of claim 3, wherein the correction of the three-dimensional image of the surface of the object further includes:
performing an elastic warping of the reconstruction of the three-dimensional image of the surface of the object as a function of the error set.

5. The endoscopic system (10) of claim 1, wherein the at least one distance sensor (22) is operable to provide a measurement of any pressure being exerted by the object on the at least one distance sensor (22).

6. The endoscopic system (10) of claim 1, wherein the at least one distance sensor (22) includes at least one of an ultrasound transducer element (43) for transmitting and receiving ultrasound signals having a time of flight that is indicative of the distance from the endoscope (22) to the object.

7. The endoscopic system (10) of claim 1, wherein the at least one distance sensor (22) includes at least one of an ultrasound transducer array (42) for transmitting and receiving ultrasound signals having a time of flight that is indicative of the distance from the endoscope (22) to the object.

8. The endoscopic system (10) of claim 1, wherein the at least one distance sensor (22) is piezoelectric ceramic transducer.

9. The endoscopic system (10) of claim 1, wherein the at least one distance sensor (22) is single crystal transducer.

10. The endoscopic system (10) of claim 1, wherein the at least one distance sensor (22) is piezoelectric thin micro-machined transducer.

11. The endoscopic system (10) of claim 1, wherein the at least one distance sensor (22) is built using capacitive micro-machining

12. The endoscopic system (10) of claim 1,
wherein the endoscope (20) further includes an imaging device (51) on a top distal end of a shaft of endoscope (20); and
wherein the at least one distance sensor (22) includes an ultrasound linear element (52) encircling the imaging device (51).

13. The endoscopic system (10) of claim 1, the at least one wherein distance sensor (22) includes a plurality of sensor elements serving as a phase-array for beam-forming and beam-steering.

14. An endoscopic method (60), comprising:
controlling an endoscopic robot (31) to advance an endoscope (20) to a target location within an anatomical region of a body;
generating a plurality of monocular endoscopic images (80) of the anatomical region (71) as the endoscope (20) is advanced to the target location by the endoscopic robot (31);
generating measurements of a distance of the endoscope (20) from an object within the monocular endoscopic images (80) as the endoscope (20) is advanced to the target location by the endoscopic robot (31); and
reconstructing a three-dimensional image of a surface of the object within the monocular endoscopic images (80) as a function of the distance measurements.

15. The endoscopic method (60) of claim 14, wherein the reconstruction of the three-dimensional image of the surface of the object includes:
building a three-dimensional depth map of the object from a temporal sequence of the monocular endoscopic images (80) of the anatomical region (71); and
correcting the three-dimensional depth map of the object relative to at least two distance measurements, each distance measurement being associated with one of the monocular endoscopic images.

16. The endoscopic method (60) of claim 15, wherein the correction of the three-dimensional image of the surface of the object includes:
generating an error set representative of a comparison of the depth map to a depth of each point of a surface of the object as indicated by the at least two distance measurements.

17. The endoscopic method (60) of claim 16, wherein the correction of the three-dimensional image of the surface of the object further includes:
performing an elastic warping of the reconstruction of the three-dimensional image of the surface of the object as a function of the error set.

18. The endoscopic method (60) of claim 14, further comprising:
generating measurements of a pressure being exerted by the object on the endoscope (20).

19. An endoscopic control unit (30), comprising:
an endoscopic robot (31) for advancing an endoscope (20) to a target location within the anatomical region (71) within a body; and
a collision/avoidance detection unit (34) is operable, as the endoscope (20) is advanced to the target location by the endoscopic robot (31), to receive a plurality of monocular endoscopic images (80) of the anatomical region (71) and to receive measurements (81) of a distance of the endoscope (20) from an object within the monocular endoscopic images (80),
wherein the collision/avoidance detection unit (34) is further operable to reconstruct a three-dimensional image of a surface of the object within the monocular endoscopic images (80) as a function of the distance measurements (81).

20. The endoscopic control unit (30) of claim 19, wherein the reconstruction of the three-dimensional image of the surface of the object includes:
building a three-dimensional depth map of the object from a temporal sequence of the monocular endoscopic images (80) of the anatomical region (71); and
correcting the three-dimensional depth map of the object relative to at least two distance measurements (81), each distance measurement (81) being associated with one of the monocular endoscopic images.
